(19) United States
(12) Patent Application Publication
(10) Pub. No.: US 2025/0170163 A1
(43) Pub. Date: May 29, 2025

(54) COMPOSITIONS AND METHODS FOR REDUCING THE OCCURRENCE OF DIARRHEA BY PROMOTING BLAUTIA OBEUM IN THE GUT MICROBIOTA

(71) Applicant: SOCIETE DES PRODUITS NESTLE S.A., Vevey (CH)

(72) Inventors: SHAILLAY KUMAR DOGRA, Epalinges (CH); CLAIRE L. BOULANGE, Oron-la-Ville (CH); IVAN MONTOLIU ROURA, Lausanne 26 (CH)

(21) Appl. No.: 18/840,644

(22) PCT Filed: Feb. 23, 2023
(86) PCT No.: PCT/EP2023/054498

§ 371 Date: Aug. 22, 2024

Related U.S. Application Data
(60) Provisional application No. 63/425,209, filed on Nov. 14, 2022.
(60) Provisional application No. 63/425,431, filed on Nov. 15, 2022.

(30) Foreign Application Priority Data
Feb. 23, 2022 (EP) 22158111.9

(51) Int. Cl. A61K 31/715; A61P 1/12

(52) U.S. Cl. CPC A61K 31/715; A61P 1/12

(57) ABSTRACT

Methods and compositions for treating diarrhea, reducing the occurrence of diarrhea and/or preventing diarrhea in an infant or young child, by promoting Blautia obeum in the gut microbiota of the infant or young child. Suitably, the methods and compositions use at least one arabinoxylan or arabinoxylan-containing material selected from the group consisting of (i) whole corn, (ii) corn arabinoxylan, (iii) corn bran, (iv) corn bran arabinoxylan, (v) whole wheat, (vi) wheat arabinoxylan, (vii) wheat bran, and (viii) wheat bran arabinoxylan.

### TECHNICAL FIELD

The present disclosure relates to compositions and methods for treating diarrhea, reducing the occurrence of diarrhea and/or preventing diarrhea in an infant or young child.

### BACKGROUND

Diarrhea is a major cause of morbidity and mortality among children in the developing world and has health impacts for children more generally. For example, diarrhea may place the child at increased risk of death and disability by reducing nutrient and fluid absorption. Understanding and management of diarrhea is therefore important to improve outcomes and enhance the success of community-based programmes, for example.

The microbiome can refer to both the composition of microorganisms in an ecosystem and their “theatre of activity”, which may include their structural elements (nucleic acids, proteins, lipids, polysaccharides), metabolites (signalling molecules, toxins, organic, and inorganic molecules), and molecules produced by coexisting hosts and structured by the surrounding environmental conditions (Berg, G., et al., 2020. Microbiome, 8(1), pp. 1-22). The gut microbiome changes rapidly and dramatically in the first years of life (Dogra S K, et al. Microorganisms. 2021; 9(10):2110). For infants or young children, certain factors such as birth mode, antibiotics usage and duration of exclusive breast-feeding impact the gut microbiome. Some other factors such as living location, siblings and furry pets can also influence the infant or young child's gut microbiome (Stewart C J, et al. Nature. 2018, Ho N T, et al. Nat. Commun. 2018). Importantly, an infant or young child's gut microbiome establishment has lasting consequences on their later health (Dogra S. K., et al. Gut Microbes. 2015; 6(5):321-5).

WO2015/066625 describes a microbiome profile associated with a mature/healthy gut microbiota in a Bangladeshi infant cohort.

There is a need for further approaches and methods to reduce the incidence and episodes of diarrhea in infants or young children.

### SUMMARY

The present disclosure is based, at least in part, on the inventors' surprising determination that levels of Blautia obeum, and putative identification of signalling molecules expressed by Blautia obeum, in the gut microbiota are inversely associated with diarrhea episodes in a cohort of infants and young children. Further, the inventors have determined that the diarrhea episodes that occurred in the population were generally not a result of Vibrio cholera or Clostridioides difficile infection. Without wishing to be bound by theory, the inventors consider that the compositions and methods provided by the present disclosure may reduce the occurrence of diarrhea and/or prevent diarrhea in an infant or young child by providing protection against pathogens and/or aiding recovery from pathogens, such as enteropathogens.

Accordingly, the present disclosure provides approaches for treating diarrhea, reducing the occurrence of diarrhea and/or preventing diarrhea in an infant or young child by promoting Blautia obeum, in the gut microbiota. The compositions and methods disclosed herein may promote the abundance and/or activity of Blautia obeum in the gut microbiota. In particular, the compositions and methods disclosed herein may increase the levels of an anti-pathogenic molecule produced by Blautia obeum in the gut microbiota. By way of example, the anti-pathogenic molecule may reduce/inhibit the growth and/or activity of a pathogenic microorganism present in the gut microbiota.

Thus, a first aspect of the present disclosure provides a composition for treating diarrhea, reducing the occurrence of diarrhea and/or preventing diarrhea in an infant or young child, wherein the composition promotes Blautia obeum in the gut microbiota of the infant or young child.

The present disclosure further provides a method for treating diarrhea, reducing the occurrence of diarrhea and/or preventing of diarrhea in an infant or young child, said method comprising administering a composition which promotes Blautia obeum in gut microbiota to the infant or young child in need thereof.

The present disclosure further relates to the use of at least one arabinoxylan or arabinoxylan-containing material for treating diarrhea, reducing the occurrence of diarrhea and/or preventing diarrhea in an infant or young child subject, wherein the at least one arabinoxylan or arabinoxylan-containing material promotes Blautia obeum in the gut microbiota of the infant or young child (e.g., the at least one arabinoxylan or arabinoxylan-containing material is administered to the infant or young child in an amount effective to promote Blautia obeum in the gut microbiota of the infant or young child). The at least one arabinoxylan or arabinoxylan-containing material may be selected from the group consisting of (i) whole corn, (ii) corn arabinoxylan (e.g., isolated or purified corn arabinoxylan), (iii) corn bran (e.g., corn bran enriched in arabinoxylan), (iv) corn bran arabinoxylan (e.g., isolated or purified corn bran arabinoxylan), (v) whole wheat, (vi) wheat arabinoxylan (e.g., isolated or purified wheat arabinoxylan), (vii) wheat bran (e.g., wheat bran enriched in arabinoxylan), and (viii) wheat bran arabinoxylan (e.g., isolated or purified wheat bran arabinoxylan).

The present disclosure further relates to the use of at least one arabinoxylan or arabinoxylan-containing material; preferably selected from the group consisting of (i) whole corn, (ii) corn arabinoxylan, (iii) corn bran, (iv) corn bran arabinoxylan, (v) whole wheat, (vi) wheat arabinoxylan, (vii) wheat bran, and (viii) wheat bran arabinoxylan in the manufacture of a medicament for reducing the occurrence of diarrhea and/or preventing diarrhea in an infant or young child subject, wherein the at least one at least one arabinoxylan or arabinoxylan-containing material promotes Blautia obeum in the gut microbiota of the infant or young child (e.g., the medicament comprises the at least one arabinoxylan or arabinoxylan-containing material is administered to the infant or young child in an amount effective to promote Blautia obeum in the gut microbiota of the infant or young child).

Preferably the arabinoxylan or arabinoxylan-containing material is selected from whole wheat, wheat arabinoxylan, wheat bran, and wheat bran arabinoxylan. Most preferably the arabinoxylan or arabinoxylan-containing material is wheat arabinoxylan or wheat bran arabinoxylan

The present disclosure further relates to the use of dark chocolate (e.g. comprising at least 85% cocoa content) for treating diarrhea, reducing the occurrence of diarrhea and/or preventing diarrhea in an infant or young child subject, wherein the dark chocolate promotes Blautia obeum in the gut microbiota of the infant or young child. The present disclosure further relates to dark chocolate (e.g. comprising at least 85% cocoa content) for use in treating diarrhea, reducing the occurrence of diarrhea and/or preventing diarrhea in an infant or young child subject, wherein the dark chocolate promotes Blautia obeum in the gut microbiota of the infant or young child.

The present disclosure further relates to the use of dark chocolate (e.g. comprising at least 85% cocoa content) in the manufacture of a medicament for treating diarrhea, reducing the occurrence of diarrhea and/or preventing diarrhea in an infant or young child subject, wherein the dark chocolate promotes Blautia obeum in the gut microbiota of the infant or young child.

Preferably, the diarrhea is not caused by a Vibrio cholerae infection.

Suitably, the infant or young child is less than about 60 months of age, suitably less than 36 months of age, suitably from about 4 to about 36 months of age or from about 10 to about 36 months of age.

Suitably, the composition is a dietary or nutritional composition.

For example, the dietary or nutritional composition/intervention may be a food, a drink, powdered supplement, an infant or young child formula, a composition for an infant or young child that is intended to be added to or diluted with human milk, for example human milk fortifier, or food stuffs intended for consumption by an infant or young child either alone or in combination with human milk, for example complementary foods.

Suitably, the dietary or nutritional composition/intervention comprises at least one arabinoxylan or arabinoxylan-containing material; preferably selected from the group consisting of (i) whole corn, (ii) corn arabinoxylan (e.g., isolated or purified corn arabinoxylan), (iii) corn bran (e.g., corn bran enriched in arabinoxylan), (iv) corn bran arabinoxylan (e.g., isolated or purified corn bran arabinoxylan), (v) whole wheat, (vi) wheat arabinoxylan (e.g., isolated or purified wheat arabinoxylan), (vii) wheat bran (e.g., wheat bran enriched in arabinoxylan), and (viii) wheat bran arabinoxylan (e.g., isolated or purified wheat bran arabinoxylan).

Suitably, the dietary or nutritional composition/intervention comprises dark chocolate (e.g. comprising at least 85% cocoa content).

The present disclosure further provides a composition comprising Blautia obeum for treating diarrhea, reducing the occurrence of diarrhea and/or preventing diarrhea in an infant or young child, wherein the diarrhea is not caused by a Vibrio cholerae infection.

The present disclosure further provides a method for predicting if an infant or young child is at risk of diarrhea, said method comprising determining the level of Blautia obeum in one or more samples obtained from the infant or young child.

### DESCRIPTION OF DRAWINGS

FIG. 1—Schematic of Microbiota and Health study

FIG. 2—Distribution of diarrhea episodes in Microbiota and Health study. Healthy vs unhealthy diarrhea outcomes are defined by a cumulative score of diarrhea prevalence in 0-24 months of age; with 0 diarrhea episodes till 24 months of age defined as the healthy group and 3+diarrhea episodes till 24 months of age defined as the unhealthy group of infants.

FIG. 3—Differences in Blautia obeum in gut microbiome of children with 0 (healthy) vs 3+ (unhealthy) diarrhea episodes in 0-24 months (cumulative scores). (A) Average profiles with confidence intervals at 95%. (B) Barplots with confidence intervals at the most discriminant time point (18 months).

FIG. 4—Differences in Blautia obeum in gut microbiome of children with 0 (healthy) vs 3+ (unhealthy) diarrhea outcomes in 10-24 months (cumulative scores). (A) Lowess fit, n=100 permutations. (B) Binomial fit, n=1000 permutations. Time-intervals with statistically significant differences, as determined by a permutation test, are indicated in grey shades (Metwally A A, et al. Microbiome. 2018).

FIG. 5—An example of a RandomForest cross-sectional model to determine if levels of Blautia obeum are predictive of risk of diarrhea.

FIG. 6—A preferred structure of wheat arabinoxylan used in the compositions and methods disclosed herein.

FIG. 7—The impact on pH for infants at 12 months old (n=6)) for specific fibres compared to a no substrate control (NSC) and a reference prebiotic (IN). Samples were collected after 24 h of simulated colonic incubations. Statistically significant differences compared to the NSC are visualized via * (0.1<padjusted<0.2), ** (0.05<padjusted<0.1) or *** (padjusted<0.05), while differences compared to IN are indicated with $/$$/$$$.

FIG. 8—The impact on total SCFA (Short Chain Fatty Acids) for infants at 12 months old (n=6)) for specific fibres compared to a no substrate control (NSC) and a reference prebiotic (IN). Samples were collected after 24 h of simulated colonic incubations. Statistically significant differences compared to the NSC are visualized via * (0.1<padjusted<0.2), ** (0.05<padjusted<0.1) or *** (padjusted<0.05), while differences compared to IN are indicated with $/$$/$$$.

FIG. 9—The impact on acetate for infants at 12 months old (n=6)) for specific fibres compared to a no substrate control (NSC) and a reference prebiotic (IN). Samples were collected after 24 h of simulated colonic incubations. Statistically significant differences compared to the NSC are visualized via * (0.1<padjusted<0.2), ** (0.05<padjusted<0.1) or *** (padjusted<0.05), while differences compared to IN are indicated with $/$$/$$$.

FIG. 10—The impact on propionate for infants at 12 months old (n=6)) for specific fibres compared to a no substrate control (NSC) and a reference prebiotic (IN). Samples were collected after 24h of simulated colonic incubations. Statistically significant differences compared to the NSC are visualized via * (0.1<padjusted<0.2), ** (0.05<padjusted<0.1) or *** (padjusted<0.05), while differences compared to IN are indicated with $/$$/$$$.

### DETAILED DESCRIPTION

Various preferred features and embodiments of the present disclosure will now be described by way of non-limiting examples. The skilled person will understand that they can combine all features of the disclosure disclosed herein without departing from the scope of the disclosure as disclosed.

It must be noted that as used herein and in the appended claims, the singular forms “a”, “an”, and “the” include plural referents unless the context clearly dictates otherwise.

The terms “comprising”, “comprises” and “comprised of”' as used herein are synonymous with “including”, “includes” or “containing”, “contains”, and are inclusive or open-ended and do not exclude additional, non-recited members, elements or method steps. The terms “comprising”, “comprises” and “comprised of” also include the term “consisting of”.

Numeric ranges are inclusive of the numbers defining the range.

The terms “at least one of” and “and/or” used in the respective context of “at least one of X or Y” and “X and/or Y” should be interpreted as “X,” or “Y,” or “X and Y.” For example, “at least one at least one arabinoxylan or arabinoxylan-containing material” and “arabinoxylan and/or arabinoxylan-containing material” should be interpreted as “arabinoxylan,” or “arabinoxylan-containing material,” or “both arabinoxylan and arabinoxylan-containing material.”

“Prevention” includes reduction of risk, incidence and/or severity of a condition or disorder. The terms “treatment” and “treat” include both prophylactic or preventive treatment (that prevent and/or slow the development of a targeted pathologic condition or disorder) and curative, therapeutic or disease-modifying treatment, including therapeutic measures that cure, slow down, lessen symptoms of, and/or halt progression of a diagnosed pathologic condition or disorder; and treatment of patients at risk of contracting a disease or suspected to have contracted a disease, as well as patients who are ill or have been diagnosed as suffering from a disease or medical condition. The terms “treatment” and “treat” do not necessarily imply that a subject is treated until total recovery. The terms “treatment” and “treat” also refer to the maintenance and/or promotion of health in an individual not suffering from a disease but who may be susceptible to the development of an unhealthy condition. The terms “treatment” and “treat” are also intended to include the potentiation or otherwise enhancement of one or more primary prophylactic or therapeutic measures. As non-limiting examples, a treatment can be performed by a patient, a caregiver, a doctor, a nurse, or another healthcare professional.

As used herein, a prophylactically or therapeutically “effective amount” is an amount that prevents a deficiency, treats a disease or medical condition in an individual, or, more generally, reduces symptoms, manages progression of the disease, or provides a nutritional, physiological, or medical benefit to the individual. The relative terms “improved,” “increased,” “enhanced,” “promoted” and the like refer to the effects for Blautia obeum in the gut microbiota of the infant or young child, from administration of the composition comprising at least one arabinoxylan or arabinoxylan-containing material; preferably selected from the group consisting of (i) whole corn, (ii) corn arabinoxylan, (iii) corn bran, (iv) corn bran arabinoxylan, (v) whole wheat, (vi) wheat arabinoxylan, (vii) wheat bran, and (viii) wheat bran arabinoxylan, relative to a composition without the arabinoxylan or arabinoxylan-containing material or with less of the arabinoxylan or arabinoxylan-containing material, but otherwise identical.

The publications discussed herein are provided solely for their disclosure prior to the filing date of the present application. Nothing herein is to be construed as an admission that such publications constitute prior art to the claims appended hereto.

The methods and systems disclosed herein can be used by doctors, health-care professionals, lab technicians, infant or young child care providers and so on.

### Composition

The composition may be suitable for or may suitably be administered to an infant or young child in any suitable form such as a nutritional composition in a dosage unit (for example a tablet, a capsule, a sachet of powder, etc). The composition may be in powder, semi-liquid or liquid form. The composition may be added to a nutritional composition, an infant formula, a food composition, a supplement for infant or young child, a baby food, a follow-up formula, a growing-up milk, an infant or young child cereal or a fortifier. In some embodiments, the composition of the present disclosure is an infant formula, a baby food, an infant or young child cereal, a growing-up milk, a supplement or fortifier that may be intended for an infant or young child.

The expressions “complementary feeding period”, “complementary period”, “transitional period”, “transitional feeding period” and “weaning period” can be interchangeably used and refer to the period during which the milk, either breast milk or formula, is substituted by other foods in the diet of an infant or young child. The infant or young child is typically moved or transitioned gradually from exclusive milk-feeding, either breast feeding or formula feeding, to mixed diet comprising milk and/or solid foods. The transitional period depends on the infant or young child but typically falls between about 4 months and about 18 months of age, such as between about 6 and about 18 months of age, but can in some instances extend up to about 24 months or more. For humans, the weaning period typically starts between 4 and 6 months of age and is considered completed once the infant or young child is no longer fed with breast milk or infant formula, typically at about 24 months of age. In some embodiments, the weaning period is between 4 and 24 months.

Suitably, the composition is a dietary composition or nutritional composition.

The expressions “dietary composition” or “nutritional composition” refer to any kind of composition or formulation that provides a nutritional benefit to an individual and that may be safely consumed by a human or an animal. Said nutritional composition may be in solid (e.g. powder), semi-solid or liquid form and may comprise one or more macronutrients, micronutrients, food additives, water, etc. For instance, the nutritional composition may comprise the following macronutrients: a source of proteins, a source of lipids, a source of carbohydrates and any combination thereof. Furthermore, the nutritional composition may comprise the following micronutrients: vitamins, minerals, fibers, phytochemicals, antioxidants, prebiotics, probiotics, and any combination thereof. The composition may also contain food additives such as stabilizers (when provided in solid form) or emulsifiers (when provided in liquid form). The amount of the various ingredients can be expressed in g/100 g of composition on a dry weight basis when it is in a solid form, e.g. a powder, or as a concentration in g/L of the composition when it refers to a liquid form (the latter also encompasses a liquid composition that may be obtained from a powder after reconstitution in a liquid such as milk, water, e.g. a reconstituted infant or young child formula or follow-on/follow-up formula or infant or young child cereal product or any other formulation designed for infant or young child or young child nutrition). Generally, the nutritional composition can be formulated to be taken enterally, orally, parenterally, or intravenously, and it usually includes one of more nutrients selected from: a lipid or fat source, a protein source and a carbohydrate source. Preferably, the nutritional composition is for oral adminsitration.

In a particular embodiment, the composition of the present disclosure is a “synthetic nutritional composition”. The expression “synthetic nutritional composition” means a mixture obtained by chemical and/or biological means.

The expression “infant formula” as used herein refers to a foodstuff intended for particular nutritional use by infant during the first months of life and satisfying by itself the nutritional requirements of this category of person (Article 2 (c) of the European Commission Directive 91/321/EEC 2006/141/EC of 22 Dec. 2006 on infant formulae and follow-on formulae). It also refers to a nutritional composition intended for infant or young child and as defined in Codex Alimentarius (Codex STAN 72-1981) and Infant Specialities (incl. Food for Special Medical Purpose). The expression “infant formula” encompasses both “starter infant formula” and “follow-up formula” or “follow-on formula”.

A “follow-up formula” or “follow-on formula” is generally given from the 6th month onwards. It constitutes the principal liquid element in the progressively diversified diet of this category of person.

The expression “baby food” means a foodstuff intended for particular nutritional use by infants or young children during the first years of life.

The expression “infant or young child cereal composition” means a foodstuff intended for particular nutritional use by infants or young children during the first years of life.

The expression “growing-up milk” (or GUM) refers to a milk-based drink generally with added vitamins and minerals, that is intended for young children or children.

The “fortifier” may be a liquid or solid nutritional composition suitable for fortifying or mixing with human milk, infant or young child formula, or growing-up milk. Accordingly, the fortifier can be administered after dissolution in human breast milk, in infant or young child formula, in growing-up milk or in human breast milk fortified with other nutrients or otherwise it can be administered as a stand-alone composition. When administered as a stand-alone composition, the milk fortifier can be also identified as being a “supplement”.

Suitably, the composition may comprise a prebiotic.

The term “prebiotic” means non-digestible carbohydrates that beneficially affect the host by selectively stimulating the growth and/or the activity of healthy bacteria in the colon of humans (Gibson G R, et al. Nat Rev Gastroenterol Hepatol. 2017).

Suitably, the prebiotic is provided in the form of dietary fibers. For example, the dietary fibers may be prebiotic fibers.

Suitably, the prebiotic may be comprised in an ingredient, for example a dietary ingredient.

The ingredient may be selected from the group consisting of purified polysaccharide or purified oligosaccharide, a dietary fiber ingredient, a semi-purified food ingredient, a raw food ingredient, a food additive, a human milk oligosaccharide (HMO), a semi-purified or purified peptido-glycan.

The semi-purified food ingredient may be a fruit, vegetable or cereal extract.

The raw food ingredient may be a fruit, vegetable or cereal.

The food additive may be a gaur gum or gum arabic.

Suitably, the prebiotic composition comprises a prebiotic (e.g. comprised in an ingredient or fibre) which promotes Blautia obeum in the gut microbiota.

For example, the composition may comprise at least one arabinoxylan or arabinoxylan-containing material; preferably selected from the group consisting of (i) whole corn, (ii) corn arabinoxylan (e.g., isolated or purified corn arabinoxylan), (iii) corn bran (e.g., corn bran enriched in arabinoxylan), (iv) corn bran arabinoxylan (e.g., isolated or purified corn bran arabinoxylan), (v) whole wheat, (vi) wheat arabinoxylan (e.g., isolated or purified wheat arabinoxylan), (vii) wheat bran (e.g., wheat bran enriched in arabinoxylan), and (viii) wheat bran arabinoxylan (e.g., isolated or purified wheat bran arabinoxylan).

Arabinoxylans are non-starch polysaccharides present in the cell walls of plant tissues and are the main non-starch polysaccharide in cereals and grasses.

Arabinoxylan may refer to a polysaccharide comprising arabinose and xylose. Arabinoxylan may refer to a polysaccharide comprising a linear backbone of xylan with arabinose side residues. Arabinoxylan may refer to a polysaccharide comprising a linear backbone of xylan with complex side chains which comprise arabinose. Arabinoxylan may refer to a polysaccharide comprising a linear β-(1-4) linked xylan backbone with α-l-arabinofuranose side residues linked via α-(1-3) and/or α-(1-2) linkages. Arabinoxylan may comprise side chains with additional sugars such as xylose, galactose and/or glucaronic acid. Arabinoxylan may comprise side chains that are highly branched and/or complex, and/or that are simple branched.

Suitably, the arabinoxylan may be a wheat, corn or barley arabinoxylan.

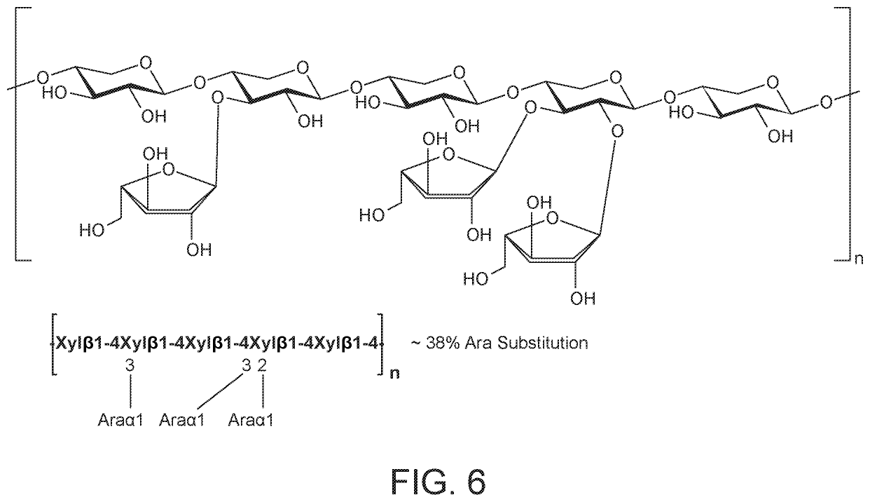

By way of example, corn and corn bran arabinoxylan have been shown to have a beneficial effect on the growth and/or survival of Bluatia or Blautia obeum (see Vatanen et al.; Cell; 2016; 165(4); 842-853 and Nguyen et al.; Microbiome; 2020l 8:118). Corn bran arabinoxylan typically comprises xylose and arabinose, in particular a backbone of linear xylose with arabinose forming branching points. The arabinoxylan may further comprise galactose. The arabinoxylan may be an extract, for example, an alkali-extract from corn, corn bran, wheat, or wheat bran. An illustrative corn bran arabinoxylan fiber is BIO-FIBER GUM (Agrifiber Holdings LLC, Illinois, USA). An illustrative wheat bran arabinoxylan is shown in FIG. 6.

As noted above, some embodiments use corn bran and/or wheat bran, which may be sifted out and/or sieved out from the other parts of the corn or wheat, and can still bring the benefits disclosed herein because the corn bran and wheat bran is enriched in arabinoxylan. Notably, refined grain does not contain arabinoxylan (or at least does not contain sufficient amounts of arabinoxylan to achieve the benefits disclosed herein). In some embodiments, corn arabinoxylan, corn bran arabinoxylan, wheat arabinoxylan or wheat bran arabinoxylan may be in a purified/extracted form, for example as available as a commercial supplier product.

Preferably the arabinoxylan or arabinoxylan-containing material is selected from whole wheat, wheat arabinoxylan, wheat bran, and wheat bran arabinoxylan. Most preferably the arabinoxylan or arabinoxylan-containing material is wheat arabinoxylan or wheat bran arabinoxylan

Additionally or alternatively to at least one arabinoxylan or arabinoxylan-containing material, the composition may comprise dark chocolate (e.g. comprising at least 85% cocoa content). By way of example, dark chocolate (e.g. comprising at least about 85% cocoa content) has been shown to have a beneficial effect on the growth and/or survival of Blautia obeum (see Shin J-H, et al.; J Nutr Biochem; 2022). The dark chocolate may be any suitable dark chocolate product (e.g. comprising at least about 85% cocoa content). The cocoa may be in the form of cocoa powder. The dark chocolate may comprise fat, protein, sugar and polyphenol.

The nutritional composition may comprise oligosaccharide(s) (e.g. human milk oligosaccharides) and/or at least a fiber(s) and/or at least a precursor(s) thereof. The oligosaccharide and/or fiber and/or precursor thereof may be selected from the list comprising galacto-oligosaccharides (GOS), fructo-oligosaccharides (FOS), inulin, xylooligosaccharides (XOS), polydextrose and any combination thereof. They may be in an amount between 0 and 10% by weight of composition. In a particular embodiment, the nutritional composition can also contain at least one BMO (bovine milk oligosaccharide).

The nutritional composition may comprise a protein source. The protein can be in an amount of from 1.6 to 3 g per 100 kcal.

Protein sources based on whey, casein and mixtures thereof may be used as well as protein sources based on soy. As far as whey proteins are concerned, the protein source may be based on acid whey or sweet whey or mixtures thereof and may include alpha-lactalbumin and beta-lactoglobulin in any desired proportions.

In some embodiments the protein source is whey predominant (i.e. more than 50% of proteins are coming from whey proteins, such as 60% or 70%). The proteins may be intact or hydrolysed or a mixture of intact and hydrolysed proteins. By the term “intact” is meant that the main part of the proteins are intact, i.e. the molecular structure is not altered, for example at least 80% of the proteins are not altered, such as at least 85% of the proteins are not altered, preferably at least 90% of the proteins are not altered, even more preferably at least 95% of the proteins are not altered, such as at least 98% of the proteins are not altered. In a particular embodiment, 100% of the proteins are not altered.

In one particular embodiment the proteins of the nutritional composition are hydrolyzed, fully hydrolyzed or partially hydrolyzed. The degree of hydrolysis (DH) of the protein can be between 8 and 40, or between 20 and 60 or between 20 and 80 or more than 10, 20, 40, 60, 80 or 90. The protein component can alternatively be replaced by a mixture or synthetic amino acid, for example for preterm or low birth weight infants.

The term “hydrolysed” means in the context of the present disclosure a protein which has been hydrolysed or broken down into its component amino acids. The proteins may be either fully or partially hydrolysed. It may be desirable to supply partially hydrolysed proteins (degree of hydrolysis between 2 and 20%), for example for infants or young children believed to be at risk of developing cow's milk allergy. If hydrolysed proteins are required, the hydrolysis process may be carried out as desired and as is known in the art. For example, whey protein hydrolysates may be prepared by enzymatically hydrolysing the whey fraction in one or more steps. If the whey fraction used as the starting material is substantially lactose free, it is found that the protein suffers much less lysine blockage during the hydrolysis process. This enables the extent of lysine blockage to be reduced from about 15% by weight of total lysine to less than about 10% by weight of lysine; for example about 7% by weight of lysine which greatly improves the nutritional quality of the protein source.

In an embodiment of the disclosure at least 70% of the proteins are hydrolysed, for example at least 80% of the proteins are hydrolysed, such as at least 85% of the proteins are hydrolysed, or at least 90%, 95%, 98% of the proteins are hydrolysed. In a particular embodiment, 100% of the proteins are hydrolysed.

The nutritional composition may contain a carbohydrate source. This is particularly preferable in the case where the nutritional composition is an infant formula. In this case, any carbohydrate source conventionally found in infant formulae such as lactose, sucrose, saccharose, maltodextrin, starch and mixtures thereof may be used although one of the preferred sources of carbohydrates is lactose.

The nutritional composition may contain a source of lipids. This is particularly relevant if the nutritional composition is an infant formula. In this case, the lipid source may be any lipid or fat which is suitable for use in infant formulae. Some suitable fat sources include palm oil, structured triglyceride oil, high oleic sunflower oil and high oleic safflower oil, medium-chain-triglyceride oil. The essential fatty acids linoleic and a-linolenic acid may also be added, as well small amounts of oils containing high quantities of preformed arachidonic acid and docosahexaenoic acid such as fish oils or microbial oils. The fat source may have a ratio of n-6 to n-3 fatty acids of about 5:1 to about 15:1; for example about 8:1 to about 10:1.

The nutritional composition may also contain vitamins and minerals understood to be essential in the daily diet and in nutritionally significant amounts. Minimum requirements have been established for certain vitamins and minerals. Examples of minerals, vitamins and other nutrients optionally present in the composition of the disclosure include vitamin A, vitamin B1, vitamin B2, vitamin B6, vitamin B12, vitamin E, vitamin K, vitamin C, vitamin D, folic acid, inositol, niacin, biotin, pantothenic acid, choline, calcium, phosphorous, iodine, iron, magnesium, copper, zinc, manganese, chlorine, potassium, sodium, selenium, chromium, molybdenum, taurine, and L-carnitine. Minerals are usually added in salt form. The presence and amounts of specific minerals and other vitamins will vary depending on the intended population. If necessary, the nutritional composition of the disclosure may contain emulsifiers and stabilisers such as soy, lecithin, citric acid esters of mono-and diglycerides, and the like.

The nutritional composition may also contain other substances which may have a beneficial effect such as lactoferrin, nucleotides, nucleosides, and the like.

The nutritional composition may be prepared in any suitable manner. A composition will now be described by way of example.

For example, a formula such as an infant formula may be prepared by blending together the protein source, the carbohydrate source and the fat source in appropriate proportions. The at least one arabinoxylan or arabinoxylan-containing material, as described herein, may also be added. If used, the emulsifiers may be included at this point. The vitamins and minerals may be added at this point but they are usually added later to avoid thermal degradation. Any lipophilic vitamins, emulsifiers and the like may be dissolved into the fat source prior to blending. Water, preferably water which has been subjected to reverse osmosis, may then be mixed in to form a liquid mixture. The temperature of the water is conveniently in the range between about 50° C. and about 80° C. to aid dispersal of the ingredients. Commercially available liquefiers may be used to form the liquid mixture.

The liquid mixture is then homogenised.

The liquid mixture may then be thermally treated to reduce bacterial loads, by rapidly heating the liquid mixture to a temperature in the range between about 80° C. and about 150° C. for a duration between about 5 seconds and about 5 minutes, for example. This may be carried out by means of steam injection, an autoclave or a heat exchanger, for example a plate heat exchanger.

Then, the liquid mixture may be cooled to between about 60° C. and about 85° C. for example by flash cooling. The liquid mixture may then be again homogenised, for example in two stages between about 10 MPa and about 30 MPa in the first stage and between about 2 MPa and about 10 MPa in the second stage. The homogenised mixture may then be further cooled to add any heat sensitive components, such as vitamins and minerals. The pH and solids content of the homogenised mixture are conveniently adjusted at this point.

If the final product is to be a powder, the homogenised mixture is transferred to a suitable drying apparatus such as a spray dryer or freeze dryer and converted to powder. The powder should have a moisture content of less than about 5% by weight. The mixture may be spray-dried or freeze-dried.

If a liquid composition is preferred, the homogenised mixture may be sterilised then aseptically filled into suitable containers or may be first filled into the containers and then retorted.

The nutritional composition can be for example given immediately after birth of the infant. The nutritional composition of the disclosure can also be given during the first week of life of the infant or young child, or during the first 2 weeks of life, or during the first 3 weeks of life, or during the first month of life, or during the first 2 months of life, or during the first 3 months of life, or during the first 4 months of life, or during the first 6 months of life, or during the first 8 months of life, or during the first 10 months of life, or during the first year of life, or during the first two years of life or even more. In some particularly advantageous embodiments of the disclosure, the composition is given (or administered) to an infant or young child from about 6 months of birth of said infant or young child. For example, the composition may be given from about 6 months, about 10 months, about 12 months, about 14 months, about 16 months, about 20 months, about 24 months or about 36 months from birth.

Suitably, the composition is given (or administered) to an infant or young child from about 10 months of birth of said infant or young child.

Suitably, the composition may be administered to an infant or young child from about 6 to about 60 months of age, from about 6 to about 48 months of age, from about 6 to about 36 months of age. Suitably, the composition may be administered to an infant or young child from about 10 to about 60 months of age, from about 10 to about 48 months of age, or from about 10 to about 36 months of age.

In some embodiments, the nutritional composition is given to the infant or young child as a supplementary composition to the mother's milk. In some embodiments the infant or young child receives the mother's milk during at least the first 2 weeks, first 1, 2, 4, or 6 months. In some embodiments the nutritional composition of the disclosure is given to the infant or young child after such period of mother's nutrition or is given together with such period of mother's milk nutrition. In another embodiment the nutritional composition is given to the infant or young child as the sole or primary nutritional composition during at least one period of time, e.g., after the 1st, 2nd or 4th month of life, during at least 1, 2, 4 or 6 months.

Suitably, the composition may comprise a probiotic comprising Blautia obeum.

The term “probiotic” means microbial cell preparation or components of microbial cells with a beneficial effect on the health or well-being of the host (Salminen S, Ouwehand A. Benno Y. et al. “Probiotics: how should they be defined” Trends Food Sci. Technol. 1999:10 107-10; Hill C, et al. Nat Rev Gastroenterol Hepatol. 2014). The microbial cells are generally bacteria or yeasts.

The Blautia obeum may be included in the composition in, for example, an amount from about 10³ to 10¹² cfu of probiotic strain, more preferably between 10⁷ and 10¹² cfu such as between 10⁸ and 10¹⁰ cfu of probiotic strain per g of composition on a dry weight basis. In some embodiments, the Blautia obeum is viable. There may be both viable and inactivated Blautia obeum in some other embodiments.

The term “cfu” should be understood as colony forming unit.

Suitably, the present disclosure may comprise the use of a combination of a nutritional or dietary composition as described herein and a probiotic composition, as described herein e.g. a probiotic composition comprising Blautia obeum. Suitably, the composition and methods disclosed herein may comprise the use of a synbiotic. As used herein, a synbiotic may refer to a mixture comprising microorganisms and substrate(s) selectively utilized by the microorganism, preferably wherein the combination confers a health benefit on the host (see e.g. Swanson K S, et al. Nat Rev Gastroenterol Hepatol. 2020). For example, the present symbiotic may comprise arabionoxylan; preferably wheat or corn arabinoxylan, and/or dark chocolate (e.g. at least 85% cocoa content) as a prebiotic, and Blautia obeum as a probiotic.

The compositions according to the present disclosure may be administered by any suitable method. Preferably, the composition is for oral administration. Accordingly, the composition is preferably administered orally.

In some embodiments, the compositions according to the present disclosure can be for use before and/or during the weaning period. The nutritional composition can be administered (or given or fed) at an age and for a period that depends on the needs.

### Diarrhea

Diarrhea may be defined as three or more stools per day of decreased form (e.g., loose and/or water) from the normal. This may last for less than 14 days and be defined as acute diarhhea. Persistent diarrhea may defined as three or more stools per day of decreased form from the normal, lasting for more than 14 days but less than 1 month. Chronic diarrhea may be defined as three or more stools per day of decreased form from the normal, lasting for a month or more.

Associated symptoms of acute diarrhea, persistent diarrhea, and chronic diarrhea may include abdominal cramps or pain, fever, nausea, vomiting, fatigue, urgency, weight loss, and/or malnutrition. Acute diarrhea, persistent diarrhea, and chronic diarrhea are themselves symptoms. Causes of acute diarrhea are well known in the art, and non-limiting examples include a food allergy, antibiotic use, an enteropathogen infection, and radiation therapy. Causes of persistent diarrhea are well known in the art, and non-limiting examples include a food allergy, antibiotic use, an enteropathogen infection, colon resection, colon cancer, ulcerative colitis, necrotizing enterocolitis, Crohn's disease and radiation therapy. Causes of chronic diarrhea are well known in the art, and non-limiting examples include a food allergy, use of a pharmacological agent, an enteropathogen infection, colon resection, colon cancer, ulcerative colitis, necrotizing enterocolitis, Crohn's disease, and radiation therapy. Pharmacological agents known to cause diarrhea are well known in the art and may include, but are not limited to, an antibiotic, an anti-TNF agent, chemotherapy agents, antacids, proton pump inhibitors, (e.g. omeprazole, esomeprazole, lansoprazole, rabeprazole, patoprazole, cimetidine, ranitidine, naiziatidine), drugs that suppress the immune system (e.g. mycophenolate), antidepressants, blood pressure medications, digitalis, diuretics, cholesterol-lowering agents, lithium, theophylline, thyroid hormone, and colchicine.

Suitably, the diarrhea referred to herein may be caused by an enteropathogen infection.

Without wishing to be bound by theory, the inventors consider that the compositions and methods of the present disclosure may reduce the occurrence of diarrhea and/or prevent diarrhea in an infant or young child by providing protection against pathogens and/or aiding recovery from pathogens, such as enteropathogens.

The enteropathogen may be selected from the group consisting of Campylobacter jejuni, Campylobacter coli, Enterococcus faecalis, Enterococcus faecium, Escherichia coli, Salmonella enterica, Shigella flexneri, Shigella sonnei, Shigella dysenteriae, Campylobacter upsaliensis, Yersinia enterocolitica, Yersinia pseudotuberculosis, Vibrio cholerae, Vibrio parahaemolyticus, Clostridium difficile, Entamoeba histolytica, Entamoeba dispar, Endolimax nana, lodamoeba butschii, Chilomastix mesnili, Blastocystis hominis, Trichomonas hominis, Giardia intestinalis, Giardia lamblia, Cryptosporidium parvum, Isospora belli, Dientamoeba fragilis, Microsporidia, Strongyloides stercoralis, Angiostrongylus costaricensis, Schistosoma mansoni, Schistosoma japonicum, Cyclospora cayetanensis, Enterocytozoon bieneusi, Enterocytozoon helium, Encephalitozoon intestinalis, Encephalitozoon cuniculi, Ascaris lumbricoides, Trichuris tricuria, Ancylostoma duodenale, Necator americanus, Hymenolepsis nana, rotaviruses, human caliciviruses, astroviruses, and cytolomegaloviruses.

The enteropathogen may be selected from the group consisting of Trichomonas hominis, Giardia intestinalis, Giardia lamblia, Cryptosporidium parvum, Isospora belli, Dientamoeba fragilis, Microsporidia, Strongyloides stercoralis, Angiostrongylus costaricensis, Schistosoma mansoni, Schistosoma japonicum, Cyclospora cayetanensis, Enterocytozoon bieneusi, Enterocytozoon helium, Encephalitozoon intestinalis, Encephalitozoon cuniculi, Ascaris lumbricoides, Trichuris tricuria, Ancylostoma duodenale, Necator americanus, Hymenolepsis nana, rotaviruses, human caliciviruses, astroviruses, and cytolomegaloviruses.

The enteropathogen may be selected from the group consisting of Campylobacter jejuni, Campylobacter coli, Enterococcus faecalis, and Enterococcus faecium.

A subject at risk of diarrhea may be subject with a food allergy, who has been administered antibiotics, who has been treated with radiation therapy, who has been treated with a pharmacological agent (e.g. as described herein), a subject with a colon resection, colon cancer, ulcerative colitis, necrotizing enterocolitis, Crohn's disease, and/or a subject with an enteropathogen infection. A subject at risk of diarrhea may, for example, be a subject living in a geographic area with limited or no access to clean drinking water, a subject living in a geographic area that is experiencing a disease outbreak, or a subject that is exposed to others that have an acute diarrhea. A subject at risk of diarrhea may be a subject that is malnourished. Treating a subject at risk of diarrhea may reduce the occurrence or prevent diarrhea (e.g. acute diarrhea) in the subject.

The term “prevention” in connection with diarrhea refers to reducing or eliminating symptoms of diarrhea before they occur.

For example, preventing diarrhea may involve reducing in treated subjects (1) the incidence, development or formation of the diarrhea, (2) the development, duration, and/or severity of symptoms of the diarrhea (e.g. fever, bloody stools, vomiting), if the disease does develop, (3) death rates associated with the acute diarrhea, or (4) a combination thereof. For each aspect, the amount of reduction may each be about 10%, 20%, 30%, 40%, 50%, 60%, 70%, 80%, 90%, 95% or about 100% in treated subjects, as compared to untreated subjects.

The diarrhea referred to herein is preferably not caused by a Vibrio cholerae infection. Vibrio cholerae is a pathogen of the adult population and is not typically present in infants or young children. This is described, for example, in Bangladeshi cohorts (Taniuchi M, et al. J Infect Dis. 2013). Suitably, determination that a diarrhea episode is not caused by Vibrio cholerae infection may be achieved by testing a stool sample taken from the subject during a diarrhea episode-for example using metagenomics sequencing and/or 16S rRNA sequencing analysis-and determining the absence, or essential absence, of Vibrio cholera in the sample.

Suitably, the diarrhea referred to herein is not caused by a Clostridioides difficile infection. Suitably, determination that a diarrhea episode is not cause by Clostridioides difficile infection may be achieved by testing a stool sample taken from the subject during a diarrhea episode—for example using metagenomics sequencing and/or 16S rRNA sequencing analysis—and determining the absence, or essential absence, of Clostridioides difficile in the sample.

Suitably, the diarrhea referred to herein is not caused by Vibrio cholerae or Clostridioides difficile infection.

### Infant or Young child

Suitably, the infant or young child may be less than about 60 months of age. For example, the infant or young child may be less than 48 months or less than 36 months of age.

Suitably, the infant or young child may be from about 1 to 60 months, about 1 to 48 months, about 2 to 60 months, about 2 to 48 months, about 2 to 36 months, about 4 to 36 months or about 6 to 36 months of age.

For example, the infant or young child may be at least about 6 months, at least about 10 months, at least about 12 months, at least about 14 months, at least about 16 months, at least about 20 months, at least about 24 months or at least about 36 months of age.

Suitably, the infant or young child may be about 6 to about 60 months of age, about 6 to about 48 months of age, about 6 to about 36 months of age. Suitably, the infant or young child may be about 10 to about 60 months of age, about 10 to about 48 months of age, or about 10 to about 36 months of age.

The infant may be a child under the age of 12 months. The “young child” may be a child aged between one and less than five year, or between one and less than three years.

The subject may be a mammal. Preferably, the subject is a human. Unless stated otherwise, ages referred to herein are in respect of a human subject.

### Blautia obeum

Blautia species are strictly anaerobic, nonmotile, 1.0-1.5×1.0-3.0 μm in size, usually spherical or oval, and appear in pairs or strands, with most strains being sporeless. The optimum temperature and pH for most Blautia strains are 37° C. and 7.0, respectively. Carbohydrate utilization experiments have shown that all Blautia strains can use glucose, but different strains showed different abilities to use sucrose, fructose, lactose, maltose, rhamnose, and raffinose. The final products of glucose fermentation by Blautia are acetic acid, succinic acid, lactic acid, and ethanol, and the main biochemical tests have revealed negative results for lecithin, lipase, catalase, and indole.

Blautia obeum is known to metabolize fibres in the gut and produce metabolites such as propionate, thus contributing to the lowering of the pH in the colonic lumen (Lawson et al.; Int J Syst Evol Microbiol. 2015; 65:789-793 and Nguyen et al.; Microbiome; 2020l 8:118).

A type strain of Blautia obeum is ATCC 29174. A reference genome for Blautia obeum is provided by GenBank assembly accession: GCA_003457595.1.

Suitably, the Blautia obeum may comprise a 16S rRNA gene sequence with a cutoff identity value of 99% and minimum query and target coverages of 80% when compared to the 16S rRNA sequence of ATCC 29174 and/or GCA_003457595.1 using BLASTn. Suitable comparisons may be performed using known methods, as described by Maturana and Cárdenasm (Front Microbiol. 2021; 660920), for example.

Suitably, the Blautia obeum may have ANI (average nucleotide identity) of at least 95%, a TETRA (tetranucleotide frequency) of at least 0.99, and/or an AAI (average amino acid identity) of at least 95% compared to ATCC 29174 and/or GCA_003457595.1 for an entire genome dataset. Suitably, the Blautia obeum microorganism may have ANI (average nucleotide identity) of at least 95%, a TETRA (tetranucleotide frequency) of at least 0.99, and an AAI (average amino acid identity) of at least 95% compared to ATCC 29174 and/or GCA_003457595.1 for an entire genome dataset. Suitable comparisons may be performed using known methods, as described by Maturana and Cárdenasm (as above), for example.

Suitably, a Blautia obeum may be identified using a metagenomics method. Suitable metagenomics methods may be performed using shotgun sequencing data, for example. Metagenomics methods may also advantageously enable estimation of organismal relative abundance. Suitable metogenomics methods are known in the art and include MetaPhlAn et 3.0, for example (see Beghini al.; eLife 2021;10: e65088; https://huttenhower.sph.harvard.edu/metaphlan).

In some embodiments, the Blautia obeum is isolated from a human.

Suitably, to ‘promote Blautia obeum’ means to increase the absolute or relative numbers of Blautia obeum in the gut microbiota. For example, a nutritional composition may assist or support the growth and/or survival of the microorganism. Alternatively, a probiotic composition for use in the disclosure will comprise Blautia obeum, and thereby increase number of Blautia obeum within the gut microbiota.

The abundance of Blautia obeum in the gut microbiota may be determined, for example, by assessing the relative abundance of Blautia obeum in a sample from a subject using a metagenomics method as described herein.

The level of Blautia obeum may be compared to a reference value determined prior to administration of a composition as described herein. The reference value may be determined before a first administration of a composition as described herein, or after a first administration but before to a subsequent administration of a composition as described herein.

The method is typically practiced outside of the human or animal body, e.g. on a sample that was previously obtained from the subject to be tested. Preferably, the sample is a faecal sample.

By way of example, the composition may increase the abundance of Blautia obeum by at least 1.5-, 2-, 3-, 4-, 5-, 10-, 50-, or 100-fold compared to, for example, the abundance of Blautia obeum prior to administration of the composition.

Suitably, promoting Blautia obeum abundance and/or activity in the gut microbiota may increase levels of autoinducer-2 (Al-2; (3aS,6S,6aR)-2,2,6,6a-Tetrahydroxy-3a-methyltetrahydro-2H-furo[2,3-d][1,3,2]dioxaborol-2-uide) in the microbiome. Al-2 is a furanosyl borate diester and is a member of a family of signaling molecules used in quorum sensing. Al-2 arises by the reaction of 1-deoxy-3-dehydro-D-ribulose, which is produced enzymatically with boric acid. Al-2 is produced by Blautia obeum and reduces colonization and virulence of Vibrio cholera (Hsiao et al; Nature; 2014; 515 (7527): 423-426). Al-2 is known to regulate many bacteria functions, including of pathogenic species, (see Pereira C S, et al. FEMS Microbiol Rev. 2013). Levels of Al-2 may be measured using methods described in the art (see Campagna et al.; Anal. Chem. 2009, 81, 15, 6374-6381). Al-2 may also be measured by detecting a precursor, for example 4,5-Dihydroxy-2,3-pentanedione (DPD). This may be performed by—for example—LC/MS untargeted metabolomics, as per the present Examples.

Suitably, promoting Blautia obeum abundance and/or activity in the gut microbiota increases levels of nisin O (NSO) (Gherghisan-Filip C, et al. Sci Rep. 2018.). NSO is a lantibiotic (modified peptide) against multiple Clostridium species (Liu X, et al. Gut Microbes. 2021).

Suitably, promoting Blautia obeum abundance and/or activity in the gut microbiota increases its Bile Salt Hydrolase activity. This activity provides mechanism of action against V. cholerae (Alavi S, et al. Cell. 2020). C. perfringens (Liu X, et al. Gut Microbes. 2021 & Gherghisan-Filip C, et al. Sci Rep. 2018) and Clostridioides difficile infection (Mullish B H, et al. Gut. 2019.), for example.

### Microbiota and Microbiome

The “gut microbiota” may refer to the composition of microorganisms (including bacteria, archaea and fungi) that live in the digestive tract.

The term “gut microbiome” may encompass both the “gut microbiota” and their “theatre of activity”, which may include their structural elements (nucleic acids, proteins, lipids, polysaccharides), metabolites (signalling molecules, toxins, organic, and inorganic molecules), and molecules produced by coexisting hosts and structured by the surrounding environmental conditions (see e.g. Berg, G., et al., 2020. Microbiome, 8(1), pp. 1-22).

In the present disclosure, the term “gut microbiome” may therefore be used interchangeably with the term “gut microbiota”.

### Methods

The present disclosure further provides a method for predicting or assessing if an infant or young child is at risk of diarrhea, said method comprising determining the level of Blautia obeum in one or more samples obtained from the infant or young child.

Without wishing to be bound by theory, the inventors consider that an infant or young child with higher levels of Blautia obeum (e.g. abundance and/or activity) in their microbiome may have stronger immunity (e.g. greater protection against pathogens, greater recovery from pathogens).

The level of Blautia obeum may be compared to a reference value, wherein the comparison is indicative of the predicted risk of diarrhea for the infant or small child. The term reference level is synonymous with ‘control level’ and broadly includes data that the skilled person would use to facilitate the accurate interpretation of technical data.

The reference value may be based on a value (e.g. an average) of Blautia obeum in a population of infants and/or young children who are known to be at risk of diarrhea or to have experienced multiple episodes of diarrhea (for example 3 or more diarrhea episodes over a 24 month period, for example from 0-24 months of age, or over a period from 10-24 months of age). The reference value may be based on a value (e.g. an average) of Blautia obeum in a population of infants and/or young children who are known not to be at risk of diarrhea or who have not experienced multiple episodes of diarrhea (for example, they didn't have 3 or more diarrhea episodes from 0-24 months of age or from 10-24 months of age). The reference value may be based on a value (e.g. an average) of Blautia obeum in a population of infants and/or young children who are known not to be at risk of diarrhea or who have not experienced multiple episodes of diarrhea (for example, they had 0 diarrhea episodes from 0-24 months of age or from 10-24 months).

The reference level may be age matched with the test sample.

Suitably, the infant or young child may be from about 1 to 60 months, about 1 to 48 months, about 2 to 60 months, about 2 to 48 months, about 2 to 36 months, or about 4 to 36 months or about 6 to 36 months of age.

Preferably, the infant or young child may be at least 10 months of age.

For example, the infant or young child may be at least about 10 months, at least about 12 months, at least about 14 months, at least about 16 months, at least about 20 months, at least about 24 months or at least about 36 months of age.

Preferably, the infant or young child may be from about 10 to about 48 months of age, about 10 to about 36 months of age, about 10 to about 24 months of age, or about 10 to about 18 months of age.

A risk of diarrhea, as described herein, may refer to a risk of multiple episodes of diarrhea over a given time period, for example 3 or more diarrhea episodes from 0-24 months of age or from 10-24 months of age.

The present method is typically practiced outside of the human or animal body, e.g. on a sample that was previously obtained from the subject to be tested. Preferably, the sample is a faecal sample.

Suitably, the present method provides that a difference in the level of Blautia obeum in the test sample compared to the reference level is indicative of the risk of diarrhea. Suitably, the present method may provide that a difference in the level of Blautia obeum in the test sample compared to the reference level is indicative of an increased risk of diarrhea.

For example, a 1.1, 1.5, 2, 3, 4, 5, 10, 50, or 100-fold difference between the levels determined in the test sample and the reference level may be indicative of an increased risk of diarrhea.

Suitably, the method further comprises combining the level of Blautia obeum with one or more anthropometric measures.

Preferably, the diarrhea is not caused by a Vibrio cholerae infection and/or Clostridioides difficile infection.

Suitably, an infant or young child determined to be at risk of diarrhea using the present method may be treated with a composition to reduce the occurrence of diarrhea and/or prevent diarrhea according to the present disclosure.

### EMBODIMENTS

Various preferred features and embodiments of the present invention will now be described with reference to the following numbered paragraphs (paras).
1. A composition for use in reducing the occurrence of diarrhea and/or preventing diarrhea in an infant or young child, wherein the composition promotes Blautia obeum in the gut microbiota of the infant or young child.
2. The composition for use according to para 1 wherein the composition comprises at least one arabinoxylan or arabinoxylan-containing material in an amount effective to promote Blautia obeum in the gut microbiota of the infant or young child.
3. The composition for use according to para 2 wherein the arabinoxylan or arabinoxylan-containing material is selected from the group consisting of (i) whole corn, (ii) corn arabinoxylan, (iii) corn bran, (iv) corn bran arabinoxylan, (v) whole wheat, (vi) wheat arabinoxylan, (vii) wheat bran, and (viii) wheat bran arabinoxylan.
4. The composition for use according to any preceding para wherein the composition comprises a Blautia obeum microorganism.
5. A composition for treating diarrhea, reducing the occurrence of diarrhea and/or preventing diarrhea in an infant or young child, wherein the composition comprises at least one arabinoxylan or arabinoxylan-containing material in an amount effective to promote Blautia obeum in the gut microbiota of the infant or young child.
6. The composition according to para 5, wherein the composition comprises at least one arabinoxylan or arabinoxylan-containing material selected from the group consisting of (i) whole corn, (ii) corn arabinoxylan, (iii) corn bran, (iv) corn bran arabinoxylan, (v) whole wheat, (vi) wheat arabinoxylan, (vii) wheat bran, and (viii) wheat bran arabinoxylan in an amount effective to promote Blautia obeum in the gut microbiota of the infant or young child.
7. A composition for treating diarrhea, reducing the occurrence of diarrhea and/or preventing diarrhea in an infant or young child wherein the composition comprises a Blautia obeum microorganism.
8. An arabinoxylan or arabinoxylan-containing material for use in reducing the occurrence of diarrhea and/or preventing diarrhea in an infant or young child; wherein the arabinoxylan promotes Blautia obeum in the gut microbiota of the infant or young child.
9. The arabinoxylan or arabinoxylan-containing material for use according to para 8, wherein the arabinoxylan or arabinoxylan-containing material is selected from the group consisting of (i) whole corn, (ii) corn arabinoxylan, (iii) corn bran, (iv) corn bran arabinoxylan, (v) whole wheat, (vi) wheat arabinoxylan, (vii) wheat bran, and (viii) wheat bran arabinoxylan.
10. A Blautia obeum microorganism for use in reducing the occurrence of diarrhea and/or preventing diarrhea in an infant or young child.
11. The composition, arabinoxylan or arabinoxylan-containing material or Blautia obeum microorganism for use according to any preceding para wherein the diarrhea is not caused by a Vibrio cholerae.
12. The composition, arabinoxylan or arabinoxylan-containing material or Blautia obeum microorganism for use according to any preceding para wherein the infant or young child is less than about 60 months of age, suitably less than 36 months of age, suitably from about 6 to about 36 months of age.
13. The composition, arabinoxylan or arabinoxylan-containing material or Blautia obeum microorganism for use according to any preceding para wherein the infant or young child has been determined to be at risk of diarrhea episodes.
14. The composition, arabinoxylan or arabinoxylan-containing material or Blautia obeum microorganism for use according to any preceding para wherein the diarrhea is caused by at least one of an enteropathogen infection, a food allergy, or antibiotic use.
15. The composition, arabinoxylan or arabinoxylan-containing material or Blautia obeum microorganism for use according to para 14 wherein the enteropathogen is selected from the group consisting of Campylobacter jejuni, Campylobacter coli, Enterococcus faecalis, Enterococcus faecium, Escherichia coli, Salmonella enterica, Shigella flexneri, Shigella sonnei, Shigella dysenteriae, Campylobacter upsaliensis, Yersinia enterocolitica, Yersinia pseudotuberculosis, Vibrio parahaemolyticus, Clostridum difficile, Entamoeba histolytica, Entamoeba dispar, Endolimax nana, lodamoeba butschii, Chilomastix mesnili, Blastocystis hominis, Trichomonas hominis, Giardia intestinalis, Giardia lamblia, Cryptosporidium parvum, Isospora belli, Dientamoeba fragilis, Microsporidia, Strongyloides stercoralis, Angiostrongylus costaricensis, Schistosoma mansoni, Schistosoma japonicum, Cyclospora cayetanensis, Enterocytozoon bieneusi, Enterocytozoon helium, Encephalitozoon intestinalis, Encephalitozoon cuniculi, Ascaris lumbricoides, Trichuris tricuria, Ancylostoma duodenale, Necator americanus, Hymenolepsis nana, rotaviruses, human caliciviruses, astroviruses, cytolomegaloviruses, and combinations thereof.
16. The composition arabinoxylan or arabinoxylan-containing material or Blautia obeum microorganism for use according to para 15 wherein the enteropathogen is selected from the group consisting of Campylobacter jejuni, Campylobacter coli, Enterococcus faecalis, Enterococcus faecium, and combinations thereof.
17. The composition arabinoxylan or arabinoxylan-containing material or Blautia obeum microorganism for use according to para 14 wherein the enteropathogen is selected from the group consisting of Trichomonas hominis, Giardia intestinalis, Giardia lamblia, Cryptosporidium parvum, Isospora belli, Dientamoeba fragilis, Microsporidia, Strongyloides stercoralis, Angiostrongylus costaricensis, Schistosoma mansoni, Schistosoma japonicum, Cyclospora cayetanensis, Enterocytozoon bieneusi, Enterocytozoon helium, Encephalitozoon intestinalis, Encephalitozoon cuniculi, Ascaris lumbricoides, Trichuris tricuria, Ancylostoma duodenale, Necator americanus, Hymenolepsis nana, rotaviruses, human caliciviruses, astroviruses, cytolomegaloviruses, and combinations thereof.
18. The composition arabinoxylan or arabinoxylan-containing material or Blautia obeum microorganism for use according to any preceding para wherein the diarrhea is not caused by Clostridioides difficile infection.
19. The composition for use according to any preceding para wherein the composition is a dietary or nutritional composition.
20. The composition for use according to para 19 wherein the dietary or nutritional composition is selected from the group consisting of a food; a drink; a powdered supplement; an infant or young child formula; a composition for an infant or young child that is intended to be added to or diluted with human milk, for example human milk fortifier; food stuffs intended for consumption by an infant or young child either alone or in combination with human milk, for example complementary foods; and combinations thereof.
21. The composition for use according to para 19 or 20 wherein the dietary or nutritional composition further comprises dark chocolate.
22. A method for reducing the occurrence of diarrhea and/or preventing of diarrhea in an infant or young child subject, said method comprising administering a composition which promotes Blautia obeum in gut microbiota, to an infant or young child in need thereof.
23. The method according to para 22 wherein the composition comprises at least one arabinoxylan or arabinoxylan-containing material; preferably wherein the at least one arabinoxylan or arabinoxylan-containing material is selected from the group consisting of (i) whole corn, (ii) corn arabinoxylan, (iii) corn bran, (iv) corn bran arabinoxylan, (v) whole wheat, (vi) wheat arabinoxylan, (vii) wheat bran, and (viii) wheat bran arabinoxylan, in an amount effective to promote Blautia obeum in gut microbiota of the infant or young child.
24. A method for treating diarrhea, reducing the occurrence of diarrhea and/or preventing of diarrhea in an infant or young child, said method comprising administering at least one arabinoxylan or arabinoxylan-containing material or a composition comprising at least one arabinoxylan or arabinoxylan-containing material to an infant or young child in need thereof, wherein the at least one arabinoxylan or arabinoxylan-containing material is selected from the group consisting of (i) whole corn, (ii) corn arabinoxylan, (iii) corn bran, (iv) corn bran arabinoxylan, (v) whole wheat, (vi) wheat arabinoxylan, (vii) wheat bran, and (viii) wheat bran arabinoxylan in an amount effective to promote Blautia obeum in gut microbiota of the infant or young child.
25. A composition comprising a combination of a Blautia obeum probiotic and at least one arabinoxylan or arabinoxylan-containing material selected from the group consisting of (i) whole corn, (ii) corn arabinoxylan, (iii) corn bran, (iv) corn bran arabinoxylan, (v) whole wheat, (vi) wheat arabinoxylan, (vii) wheat bran, and (viii) wheat bran arabinoxylan, wherein the composition comprises the combination in an amount effective for treating diarrhea, reducing the occurrence of diarrhea and/or preventing diarrhea in an infant or young child.
26. The composition according to para 25 wherein the diarrhea is not caused by a Vibrio cholerae infection.
27. A Blautia obeum probiotic for use in reducing the occurrence of diarrhea and/or preventing diarrhea in an infant or young child, wherein the diarrhea is not caused by a Vibrio cholerae infection.
28. A method for predicting if an infant or young child is at risk of diarrhea, said method comprising determining the abundance of Blautia obeum in one or more samples obtained from the infant or young child.

### EXAMPLES

The compositions and methods according to the present disclosure will now be further described by way of examples, which are meant to serve to assist the skilled person in carrying out the disclosure and do not in any way limit the scope of the disclosure.

### Example 1: Microbiota and Health Study

The Microbiota and Health study is described in Vidal et al. (https://www.medrxiv.org/content/10.1101/19000505v1) and registered at clinicaltrials.gov as NCT02361164.

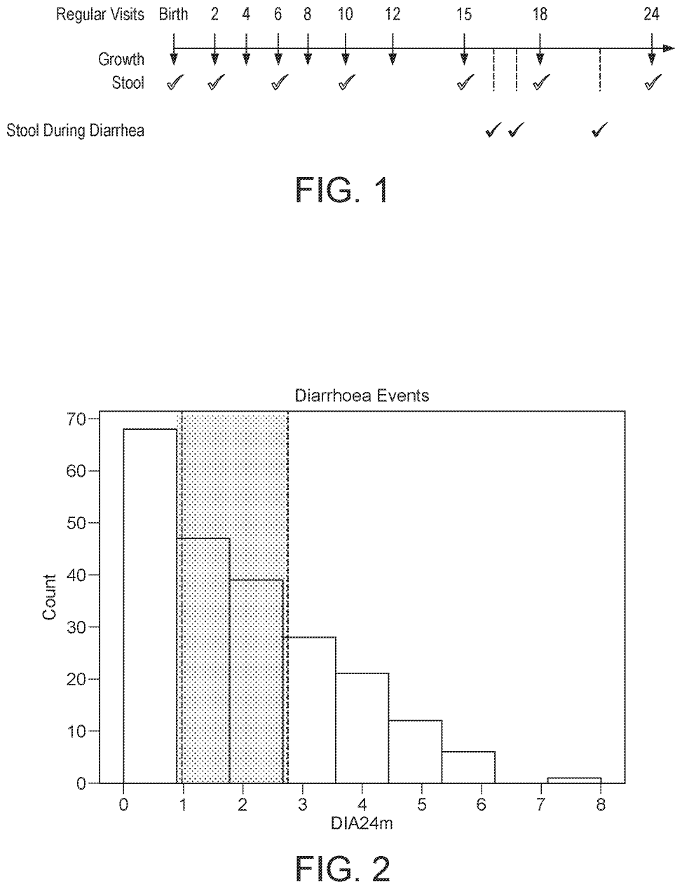

Briefly, for n=220 infant or young children, the following information was collected: Anthropometry, Diarrhea and Acute Respiratory Infections (ARI); Medication, including antibiotic use; Breastfeeding status, weaning foods; Nasopharyngeal and stool pathogens and microbiota profiles; Secretor status (FUT2, FUT3). Stool samples for microbiota analysis were collected at birth, 2 months, 6 months, 10 months, 15 months, 18 months, and 24 months. Growth outcomes were noted at birth, 2 months, 4 months, 6 months, 8 months, 10 months, 12 months, 15 months, 18 months and 24 months. See FIG. 1 for an overview.

### Example 2: Definition of a Healthy Reference Population

The profile of diarrhea episodes in the Microbiota and Health study was assessed. Healthy vs unhealthy diarrhea outcomes were assigned by a cumulative score of diarrhea incidences from 0-24 months of age; with 0 diarrhea incidences till 24 months of age defined as the healthy group and 3+diarrhea incidences till 24 months of age defined as the unhealthy group of infants (see FIG. 2).

### Example 3: Differences in Blautia obeum Between Healthy and Unhealthy

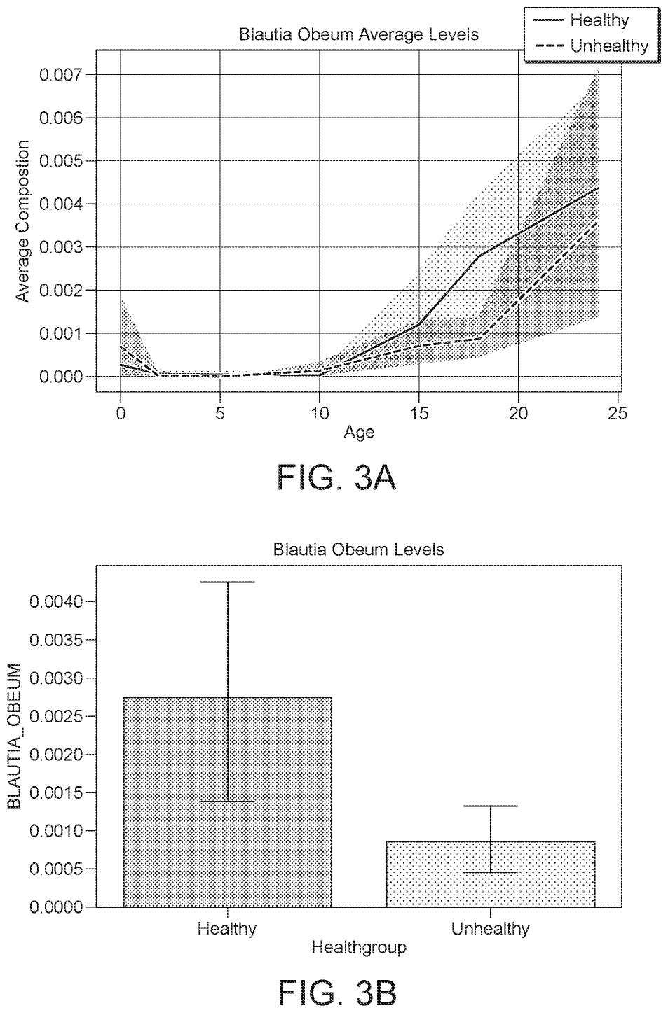

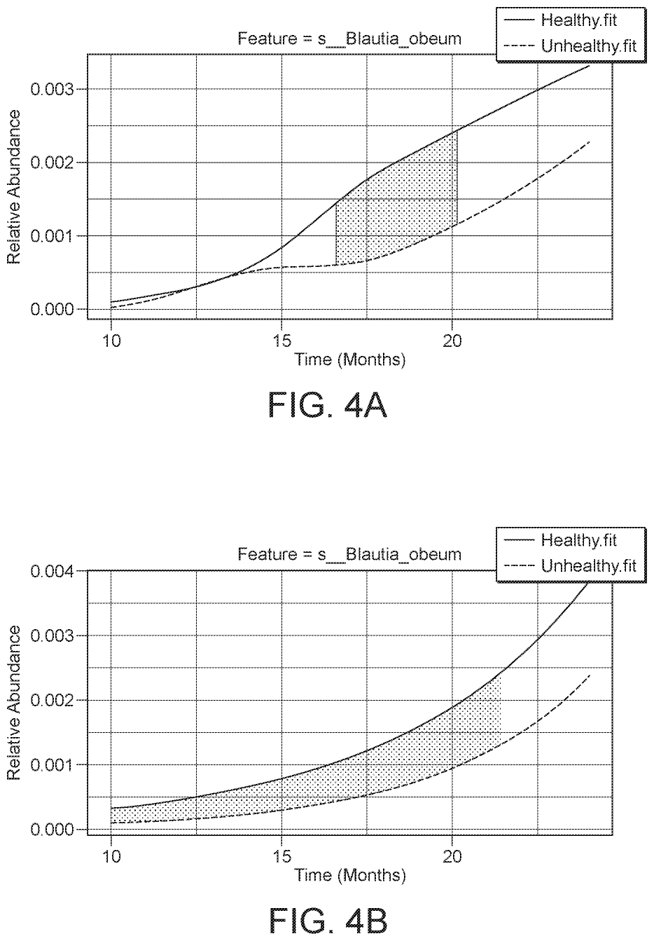

Blautia obeum levels in stool samples were associated with diarrhea outcomes of 0 vs. 3+ from 0-24 months (cumulative scores of diarrhea incidences) (see FIG. 3). Further, longitudinal, permutation based analysis using the method of Metwally A A, et al. (Microbiome; 2018; 6 (32)) comparing the abundance of Blautia obeum levels in stool samples of infant or young children with 0 diarrhea compared to 3+episodes of diarrhea (10-24 months cumulative scores) identified statistically significant differences (see FIG. 4).

In total, 56 samples were collected during an actual diarrhea episode. For these samples, which are from multiple infant or young children (n=18) at different time-points, metagenomics sequencing was performed using the MetaPhlAn 3.0 pipeline (see Beghini et al.; eLife 2021; 10: e65088; https://huttenhower.sph.harvard.edu/metaphlan).

Mainly, the pathogen Campylobacter_jejuni was identified, with 23 samples having non-zero values (minimum—0.0000256, maximum—0.32588493, median—0.0015, average—0.0333, where maximum relative abundance possible is 1.0). Other pathogens such as Campylobacter_coli (present in 3 samples), Enterococcus_faecalis (present in 1 sample), Enterococcus_faecium (present in 11 samples—minimum—0.0000437, maximum—0.110366511, median—0.0016934, average—0.020927274 relative abundance, where again maximum relative abundance possible is 1.0) were identified.

For Vibrio_cholerae most diarrhea samples had a 0.0 relative abundance value (where maximum possible value is 1.0). There were only two samples with a non-zero relative abundance value for Vibrio_cholerae (0.04785131, 0.0034564). Further, Clostridioides_difficile was 0.0 relative abundance in all these diarrhea samples.

### Example 4: Bacterial Quorum-Sensing Signal

Levels of autoinducer-2 (Al-2), a quorum sensing molecule produced by Blautia obeum, were assessed in fecal samples of the Dhaka cohort analysed by LC/MS untargeted metabolomics.

The precursor of Al-2, 4,5-Dihydroxy-2,3-pentanedione (DPD) was used as a proxy to evaluate the presence of Al-2. When using LC/MS based untargeted metabolomics, the molecules in biological samples are ionised into different molecular adducts depending on the solvent used for the molecular extraction and the ionisation mode employed during the analysis. The most common adducts are hydrogen adduct (M+H, M−H M−H−H20 (removal of water)), sodium adduct (M+Na), potassium adduct (M+K) and ammonium adduct (M+NH4+). The theoretical molecular mass of the adduct form of DPD was calculated and compared with the masses of the metabolic features detected in the fecal metabolomic dataset. The selection criteria to identify a match between the DPD adducts and the metabolic features was set to a mass difference inferior to 5 ppm.

Three metabolic features (QI31895, QI19815, QI4308) were putatively assigned as DPD as their masses matched those of the DPD adducts (M−H & M+K) with a mass difference below 1 ppm.

A linear model including time as cofactor showed a significant negative association between the metabolic feature QI31895 (nomical coeff pval=0.0055) and diarrhea incidence. The same metabolic feature was significantly lower in infants with three or more diarrhea incidences than infants with no diarrhea incidence at 18 months (nominal p-value: 0.038 Mann-Whitney U test).

### Example 5: Blautia obeum is Predictive of Diarrhea Risk

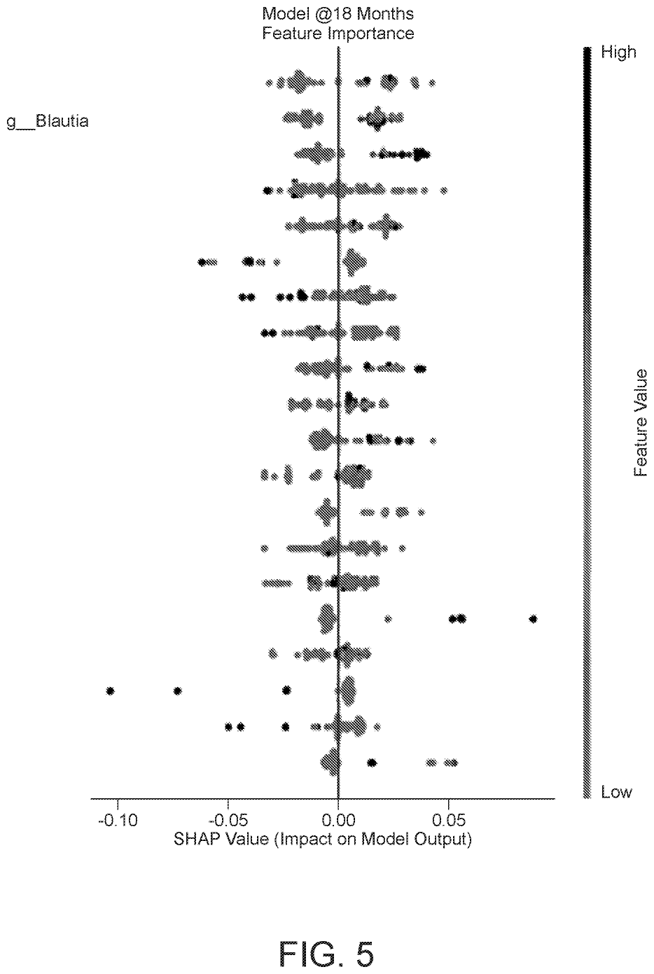

Machine-learning based models were used to determine if Blautia obeum is predictive of the occurrence of diarrhea (DIA). We computed the difference between cumulative number of DIA events at 24 months and at 10 months, as before. We stratified the infants into two groups, labelled as 0, when there are 0 DIA event and 1 when there are any DIA events. We performed a set of RandomForest cross-sectional models with parameter optimization, to detect where Blautia amounts are discriminatory in the model against DIA outcomes and then to assess its relative feature importance. In the family of models prepared, Blautia obeum was mostly considered to be in the top 15 of important features of the selected models, with F1 scores in the test set around 0.6. The best time points to predict the diarrhea state were at 15 and 18 months. An example of such a result is shown in FIG. 5 where Blautia was found to be the second most important bacteria feature to predict diarrhea outcomes at 18 months of infant age.

### Example 6: Effects of Wheat Arabinoxylan on Infant Microbiome

Samples were tested with either NSC (negative control), IN (inulin-positive control), W (wheat arabinoxylan) or C (unrelated fibre) (each product at 2 g/L).

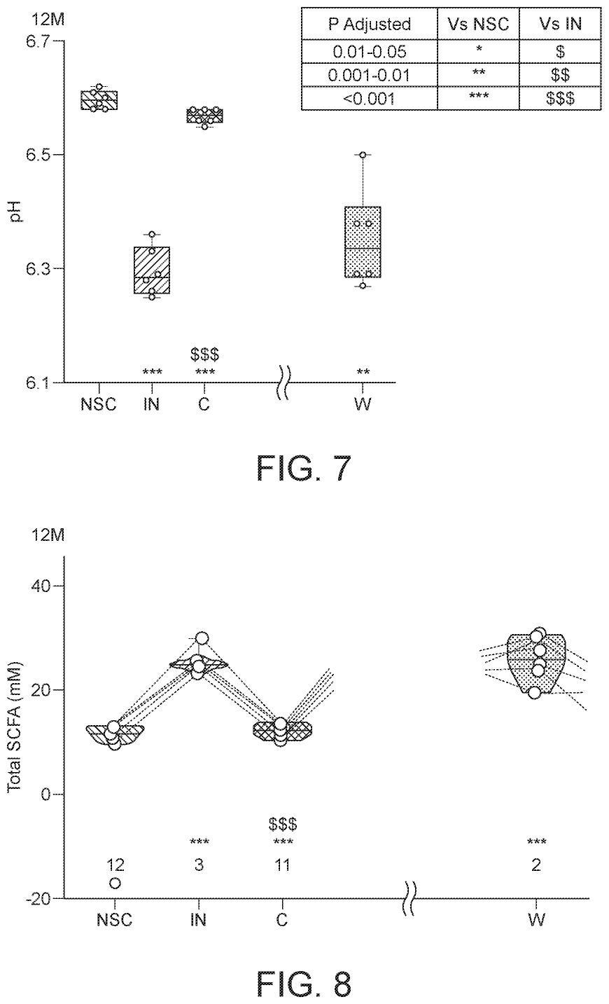

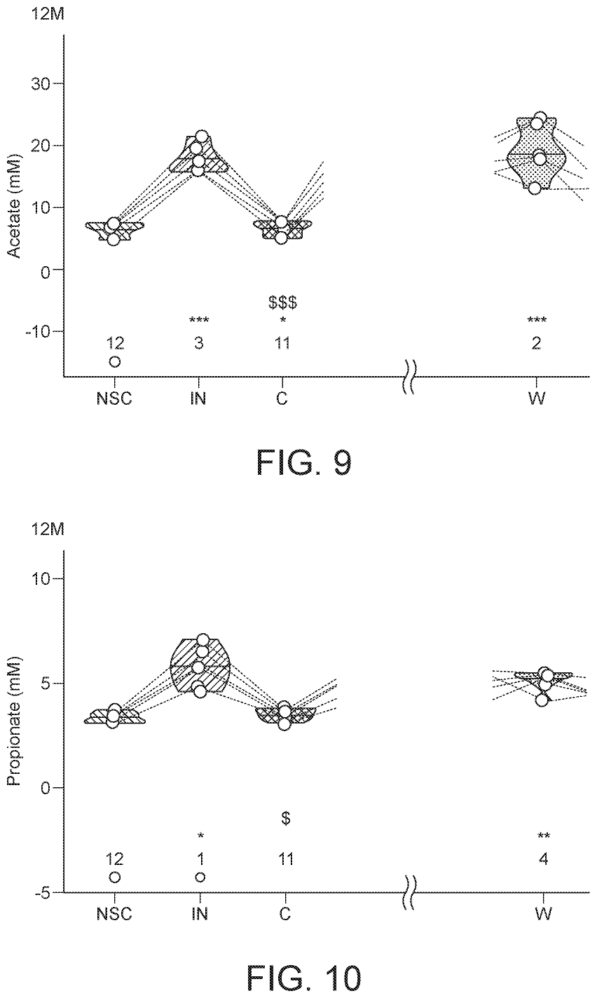

Wheat arabinoxylan significantly increased acetate (FIG. 9) and propionate (FIG. 10) (and thus also total SCFA (Short Chain Fatty Acids) (FIG. 8) levels, decreased pH (FIG. 7) and increased gas production.

Notably reduction of pH and increased propionate levels are effects that are expected with increased Blautia obeum levels.

Quantitative shallow shotgun sequencing is performed to confirm Blautia obeum levels in the respective samples.

### Materials & Methods

Faecal samples were collected according to a procedure approved by Ethics Committee of the University Hospital Ghent. The selection criteria for the donors were as follows: 6 donors of 6 (±1) months, 6 donors of 12 (±1) months, exclusively breastfed until 4 months of age and already introduced to complementary foods. The exclusion criteria antibiotic use in 90 days before sample delivery for the study and previous NEC or gut surgery. This resulted in the enrolment of 12 specific test subjects.

Colonic fermentation of the test products by gut microbioata in the faecal samples was assessed at 24 hours post-inoculation. Acetate, propionate, butyrate, valerate and branched chain fatty acids (bCFA) (sum of isobutyrate, isovalerate and isocaproate) were determined via a GC-FID approach. Lactate was measured with a high-throughput spectrophotometric method. Further, also pH and gas production were measured as key markers for microbial metabolic activity.

For the statistical evaluation of the treatment effects on fundamental fermentation parameters, cell counts, microbial diversity and microbial composition (phylum level) across infants (all 12 infants or across the 6 infants within each age group), a repeated measures ANOVA analysis was performed (˜based on paired t-testing, thus accounting for fact that values are compared between samples of a given donor). The statistical significance of the potential treatment effects was determined via Benjamini-Hochberg post hoc testing

For quantitative shallow shotgun sequencing, upon DNA extraction, standardized Illumina library preparation was performed followed by 3M total DNA sequencing.

All publications mentioned in the above specification are herein incorporated by reference. Various modifications and variations of the disclosed methods, compositions and uses of the disclosure will be apparent to the skilled person without departing from the scope and spirit of the disclosure. Although the disclosure has been disclosed in connection with specific preferred embodiments, it should be understood that the disclosure as claimed should not be unduly limited to such specific embodiments. Indeed, various modifications of the disclosed modes for carrying out the disclosure, which are obvious to the skilled person are intended to be within the scope of the following claims.

1. A method for treating diarrhea, reducing the occurrence of diarrhea and/or preventing diarrhea in an infant or young child, comprising administering a composition that promotes Blautia obeum in the gut microbiota of the infant or young child.

2. The method according to claim 1 wherein the composition comprises at least one arabinoxylan or arabinoxylan-containing material in an amount effective to promote Blautia obeum in the gut microbiota of the infant or young child.

3. A method for treating diarrhea, reducing the occurrence of diarrhea and/or preventing diarrhea in an infant or young child; comprising administering an arabinoxylan or arabinoxylan-containing material to the infant or young child wherein the arabinoxylan promotes Blautia obeum in the gut microbiota of the infant or young child.

4. The method of claim 2 wherein the arabinoxylan or arabinoxylan-containing material is selected from the group consisting of (i) whole corn, (ii) corn arabinoxylan, (iii) corn bran, (iv) corn bran arabinoxylan, (v) whole wheat, (vi) wheat arabinoxylan, (vii) wheat bran, and (viii) wheat bran arabinoxylan.

5. (canceled)

6. A method for use in treating diarrhea, reducing the occurrence of diarrhea and/or preventing diarrhea in an infant or young child comprising administering a Blautia obeum microorganism to the infant or young child.

7. The method according to claim 1 wherein the diarrhea is not caused by Vibrio cholerae.

8. The method according to claim 1 wherein the infant or young child is less than about 60 months of age.

9. The method according to claim 1 wherein the diarrhea is caused by at least one of an enteropathogen infection, a food allergy, or antibiotic use.

10. The method according to any preceding claim 1 wherein the diarrhea is not caused by Clostridioides difficile infection.

11. The method according to claim 1 wherein the composition is a dietary or nutritional composition.

12-15. (canceled)
